(19) United States
(12) Patent Application Publication
(10) Pub. No.: US 2022/0404362 A1
(43) Pub. Date: Dec. 22, 2022

(54) Methods and Systems for Detecting SARS-CoV-2 Analytes in Dried Samples

(71) Applicant: Laboratory Corporation of America Holdings, Burlington, NC (US)

(72) Inventors: Bradley B. Collier, Chapel Hill, NC (US); Russell Phillip Grant, Chapel Hill, NC (US)

(21) Appl. No.: 17/845,502

(22) Filed: Jun. 21, 2022

Related U.S. Application Data
(60) Provisional application No. 63/212,862, filed on Jun. 21, 2021.

(51) Int. Cl. G01N 33/569; G01N 33/543; B01L 3/00

(52) U.S. Cl. CPC G01N 33/56983; G01N 33/54366; B01L 3/5023; G01N 2470/04; B01L 2300/0816; B01L 2300/069

(57) ABSTRACT

Disclosed are methods and systems for detecting SARS-CoV-2 analytes in dried samples, as for example, dried blood spots. For example, disclosed is a method for measuring an analyte of interest in a dried sample comprising: (a) obtaining a dried sample from a subject; (b) extracting the analyte of interest from the dried sample; and (c) detecting the analyte of interest extracted from the dried sample. In certain embodiments, the analyte of interest is an analyte specific to SARS-CoV-2. Also, the method may include a step of determining a cutoff index (COI) indicative of whether the subject has a detectable amount of the analyte of interest and so is defined as positive, or does not contain a detected amount of the analyte of interest and so is defined as negative, or is defined as indeterminate.

### RELATED APPLICATIONS

This application claims priority to U.S. Provisional Patent Application No. 63/212,862, filed Jun. 21, 2021. The disclosure of U.S. Provisional Patent Application No. 63/212,862 is incorporated by reference in its entirety herein.

### FIELD

The present disclosure relates to methods and systems for detecting SARS-CoV-2 analytes in dried samples, as for example, dried blood spots.

### INTRODUCTION

SARS-CoV-2 is an enveloped, single-stranded RNA virus of the family Coronaviridae, genus Beta coronavirus. All coronaviruses share similarities in the organization and expression of their genome, which encodes 16 nonstructural proteins and the 4 structural proteins: spike (S), envelope (E), membrane (M), and nucleocapsid (N). Viruses of this family are of zoonotic origin. They cause disease with symptoms ranging from those of a mild common cold to more severe ones such as the Severe Acute Respiratory Syndrome (SARS), Middle East Respiratory Syndrome (MERS) and Coronavirus Disease 2019 (COVID-19). Other coronaviruses known to infect people include 229E, NL63, OC43 and HKU1. The latter are ubiquitous and infection typically causes common cold or flu-like symptoms (Su S, Wong G, Shi W, et al. Epidemiology, Genetic Recombination, and Pathogenesis of Coronaviruses. Trends Microbiol 2016; 24(6):490-502; Zhu N, Zhang D, Wang W, et al. A Novel Coronavirus from Patients with Pneumonia in China, 2019. N Engl J Med 2020; 382(8):727-733).

As the outbreak has progressed, it has become clear that the full scope of the number of people exposed to SARS-CoV-2 cannot be determined by molecular testing that is appropriate for active infections. Therefore, a widely available high throughput method for determining who has or who has not been previously infected is needed. Large population analysis is needed to determine the percent of population who have been infected to assist in policy decisions for prevention of viral spreading. One potential method for large scale sample collection while still providing safe and effective testing for patients is by lancing a finger and applying blood to a blood spot card followed by shipping to a central laboratory for testing. Dried blood spot card (DBS) collection can be performed by a trained phlebotomist or self-collected in one's home.

### SUMMARY

Disclosed are methods and systems for detecting an analyte of interest in a dried sample. In certain embodiments, the sample is a dried blood spot or a dried plasma sample. In certain embodiments, the analyte of interest is an antibody to SARS-CoV-2. The methods and systems may be embodied in a variety of ways.

For example, in certain embodiments the method may comprise measuring an analyte of interest in a dried sample comprising: (a) obtaining a dried sample from a subject; (b) extracting the analyte of interest from the dried sample; and (c) detecting the analyte of interest extracted from the dried sample. In certain embodiments, the analyte of interest is an analyte specific to SARS-CoV-2. In certain embodiments, the dried sample may be a dried blood spot or dried plasma. The method may include providing a cutoff that the sample is positive or negative, or optionally indeterminate, for the analyte of interest.

Also disclosed are systems for performing any one of the disclosed methods or steps of the disclosed methods, and a computer-program product tangibly embodied in a non-transitory machine-readable storage medium, including instructions configured to cause one or more data processors to perform processing comprising performing any of the steps of the disclosed methods or running any part of the disclosed systems.

### BRIEF DESCRIPTION OF THE FIGURES

The present disclosure may be better understood by referencing the following non-limiting figures.

FIG. 1 illustrates a method in accordance with an embodiment of the disclosure.

FIG. 2 shows a sample of dried blood spots used in accordance with an embodiment of a method or system of the disclosure.

FIG. 3 illustrates a system in accordance with an embodiment of the disclosure.

FIG. 4 illustrates a distribution of patient serum results over 8 days in accordance with an embodiment of the disclosure.

FIG. 5 illustrates a distribution of serum results for cutoff verification study in accordance with an embodiment of the disclosure.

FIG. 6 illustrates overall cutoff verification correlation results for dried blood spot (DBS) samples as compared to serum in accordance with an embodiment of the disclosure.

FIG. 7 illustrates cutoff verification correlation results for dried blood spot (DBS) samples as compared to serum zoomed into the cutoff region in accordance with an embodiment of the disclosure.

FIG. 8 illustrates correlation results overall for two different matrices in accordance with an embodiment of the disclosure.

FIG. 9 illustrates correlation results zoomed in to the cutoff region for two different matrices in accordance with an embodiment of the disclosure.

FIG. 10 illustrates quantitative correlation results for DBS anti-SARS-CoV-2 antibody levels as compared to serum anti-SARS-CoV-2 antibody levels in accordance with an embodiment of the disclosure.

FIG. 11 is an alternate illustration of quantitative correlation results for DBS anti-SARS-CoV-2 antibody levels as compared to serum anti-SARS-CoV-2 antibody levels in accordance with an embodiment of the disclosure.

### DETAILED DESCRIPTION

### Definitions

The present disclosure now will be described more fully hereinafter. The disclosure may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this disclosure belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section or as used elsewhere herein prevails over the definition that is incorporated herein by reference.

When introducing elements of the present disclosure or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the disclosure described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

“Sample” or “patient sample” or “biological sample” or “specimen” are used interchangeably herein. Non-limiting examples of liquid samples that may be dried for analysis with the disclosed systems and methods include, blood or a blood product (e.g., serum, plasma, or the like), urine, nasal swabs, a liquid biopsy sample (e.g., for the detection of cancer), or combinations thereof. The term “blood” encompasses whole blood, blood product or any fraction of blood, such as serum, plasma, buffy coat, or the like as conventionally defined. Suitable samples include those which are capable of being deposited onto a substrate for collection and drying including, but not limited to: blood, plasma, serum, urine, saliva, tear, cerebrospinal fluid, organ, hair, muscle, or other tissue samples or other liquid aspirates. In an embodiment, the sample body fluid may be separated on the substrate prior to drying. For example, blood may be deposited onto a sampling paper substrate which limits migration of red blood cells allowing for separation of the blood plasma fraction prior to drying in order to produce a dried plasma sample for analysis.

### Methods for Detection of SARS-CoV-2 Analytes in Dried Samples

Disclosed are methods and systems for detecting an analyte of interest in a dried sample. In certain embodiments, the analyte of interest is an antibody to SARS-CoV-2. For example, in certain embodiments the method may comprise a method for measuring an analyte of interest in a dried sample comprising: (a) obtaining a dried sample from a subject; (b) extracting the analyte of interest from the dried sample; and (c) detecting the analyte of interest extracted from the dried sample. In certain embodiments, the analyte of interest is an analyte specific to SARS-CoV-2. For example, the analyte of interest may be an antibody to SARS-CoV-2. In certain embodiments, the dried sample may be a dried blood spot or dried plasma. In some embodiments; disclosed are methods for measuring SARS-CoV-2 in a dried blood spot. For example, in certain embodiments the method may comprise a method for measuring an analyte specific to SARS-CoV-2 in a dried blood spot comprising: (a) obtaining a sample from a subject, the sample comprising a dried blood spot (DBS); (b) extracting an analyte specific to SARS-CoV-2 from the DBS; and (c) detecting the analyte specific to SARS-CoV-2 extracted from the DBS.

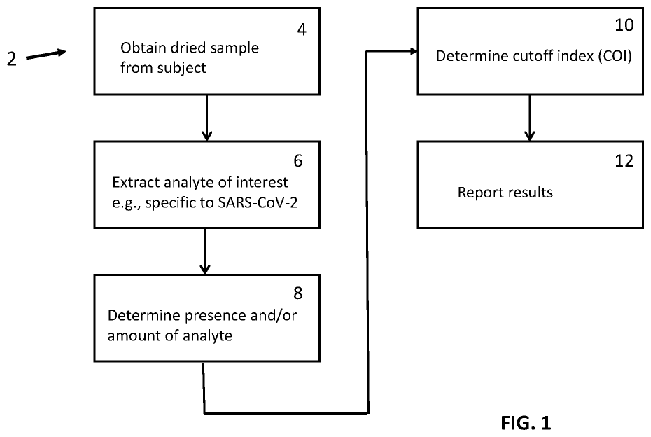

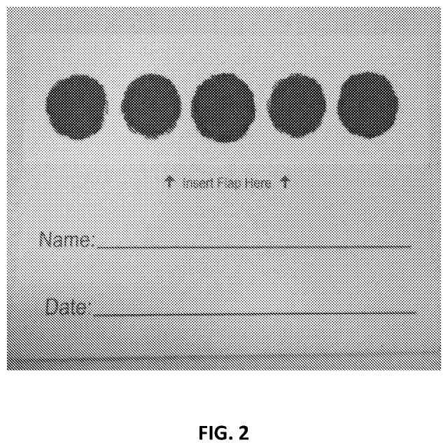

Thus, as illustrated in FIG. 1, the method (2) may comprise the step of obtaining a dried sample from a subject (4). In certain embodiments, the sample is a dried blood spot or a dried plasma sample. In an embodiment, the dried blood spot (DBS) may be obtained by a subject taking a small sample of his or her own blood. Thus, in an embodiment, the DBS may be procured by a subject in his or her own home, without the need to visit a health care professional or commercial testing site. Or, when the subject is not able to take their own sample (e.g., a child or a non-human subject) another individual may procure the DBS. In an embodiment, proper dosing of the DBS card is critical to the extraction and measurement of the sample. For example, blood (i.e. from a lanced finger) may be added dropwise to a DBS card. An example of such a card is shown in FIG. 2. In an embodiment, blood is applied to each of the 5 circles (defined by dashed lines on the card) until blood fills these predefined regions. In certain embodiments, enough sample is required to obtain two punches of approximately ¼″ diameter completely saturated with blood. Or, other sample sizes (volumes) may be used.

Generally, any type of substrate suitable for depositing a liquid sample for drying and subsequent extraction of an analyte of interest may be used. For example, Perkin Elmer 226, Whatman 903, or Eastern Business Forms 903 dried blood spot cards can be used. Blood spots may be obtained on a card and dried for a minimum for 3 hours using instructions provided with a blood collection kit. Sampling may be by a medical professional, the subject requesting testing and/or another individual with the subject's permission. In an embodiment, samples returned to laboratory can be tested up to 36 days from collection as long as sample remains in a blood sample return bag or other appropriate packaging.

The disclosed methods and systems may be used to measure a variety of analytes. As used herein, an analyte is a molecule or biological compound being analyzed either qualitatively (e.g., for identification) or quantitatively (e.g., to determine a relative or absolute amount). In an embodiment, the analyte is specific to SARS-CoV-2. For example, in some embodiments, the SARS-CoV-2 specific analyte is an antibody to SARS-CoV-2. Or, the analyte may be a protein or antigen. For example, in some embodiments, the SARS-CoV-2 specific analyte is an antigen or other protein specific to SARS-CoV-2. Or, the analyte may be a nucleic acid. For example, in some embodiments, the SARS-CoV-2 specific analyte is an nucleic acid specific to SARS-CoV-2. Or, analytes from other viruses, bacteria, or other sources or analytes of interest may be analyzed.

As shown in FIG. 1, the method may further comprise extracting the analyte of interest from the dried sample (6). For example, in certain embodiments, the method may comprise extracting the analyte specific to SARS-CoV-2 from a DBS (6). The method of extraction may be varied depending upon the technique used to measure the analyte of interest. In certain embodiments, and as discussed in detail herein, the extraction reagent and/or volume may be modified from that which is typically used as is needed to optimize recovery and measurement of the analyte of interest from a dried sample as compared to plasma, blood or other liquid samples.

As further illustrated in FIG. 1, the method may further comprise determining the presence and/or amount of the analyte in the sample (8). The disclosed methods and systems may be used with a variety of analytical techniques. In one embodiment, the analytical technique comprises a Roche Elecsys Anti-SARS-CoV-2 (ACOV2) assay as disclosed in detail herein or a similar assay. In certain embodiments, the analyte of interest is an antibody. In such embodiments, the antibody of interest may be measured using a sandwich assay. The sandwich assay may employ a first antigen labeled with a detectable moiety and a second antigen labeled with a binding agent. For example, SARS-CoV-2 antibody extracted from a dried sample, such as a DBS or dried plasma, may then be measured by detection of SARS-CoV-2 antibody bound to the first and second antigen. The detectable moiety may, in certain embodiments, comprise an electrochemical moiety such as ruthenium. Or other detectable moieties, such as radiolabels, fluorescent labels, heavy isotopes, and the like, may be used. Additionally and/or alternatively, the binding agent may comprise streptavidin. Or, other binding agents, such as secondary antibodies, receptor ligands, and the like, may be used. For example, in certain embodiments, a ruthenium labeled first antigen: SARS-CoV-2 antibody: streptavidin labeled second antigen complex is bound to a biotin labeled electrode such that application of a voltage results in a chemiluminescent emission.

As further illustrated in FIG. 1, in certain embodiments, the detected analyte is used to calculate a cutoff index (COI) indicative of whether the subject has a detectable amount of the analyte and so is defined as positive, or does not contain a detectable amount of the analyte and so is defined as negative (10). An indeterminate region may also be implemented around the pre-determined assay cutoff; such results within this region are not defined as negative or positive. Thus, the assay may be designed so that there is a COI provided for interpretation of a sample as being positive or negative for the presence (or a specified amount) of a predetermined analyte. The assay may also be designed so that there is a COI provided for interpretation of a sample as being positive, negative, or indeterminate for the presence (or a specified amount) of a predetermined analyte. In certain embodiments, the negative/positive COI is calculated to incorporate dilution of the sample that occurs during extraction of the SARS-CoV-2 specific analyte from the dried blood spot. In such embodiments, and as described in detail herein, the measured value may be divided by a fractional cutoff (which accounts for dilution) to provide a normalized value. The normalized value may, in some embodiments, be normalized to cutoff values used in serum. Or, other normalized values may be used.

Thus, the cutoff, or COI, may defined using any one of a variety of approaches. Generally, for a serology qualitative assay, results less than the cutoff are defined as negative, and results greater than or equal to the cutoff are positive. The cutoff can be any number, for example one assay may have a value of 10 and another assay may have a value of 2. Units of the cutoff can be described in terms of the analyte being measured (e.g., concentration of an antibody analyte), response of the assay, or any other unit. In some cases, units are not required for a qualitative assay.

In an embodiment, for a defined and/or commercial and/or FDA approved assay, the negative and positive cutoff is defined as 1.0. For example, for the Roche Elecsys Anti-SARS-CoV-2 assay, the negative and positive cutoff is defined as 1.0. Often in this embodiment, assay calibrators are pre-defined by the manufacturer and used to determine the assay measurement that is equivalent to 1.0.

In an embodiment, application of the Roche Elecsys Anti-SARS-CoV-2 assay to a dried sample, such as a dried blood spot, utilizes the same serum cutoff calibration. However, due to the dilutive nature of the extraction process, the cutoff value for negative and positive samples is a fraction of 1.0. In this case, and as described in more detail herein, the studies are performed to determine what that fraction of the cutoff is, and the alternate sample cutoff for the dried sample (e.g., DBS). For example, a serum sample that measures exactly 1.0 may have an equivalent DBS result of 0.1 due to the dilutive nature of extraction In this case, negative DBS samples will have a measured value of less than 0.1 and positive DBS samples will have a measured value of greater than or equal to 0.1. To simplify the interpretation of the DBS results and facilitate direct comparison to serum results, measurements may be corrected (or normalized) by dividing by the fractional cutoff. For example, a DBS sample may have a measurement of 0.25 (positive relative to the DBS fractional cutoff of 0.1). In this case, the measured value of 0.25 is divided by the fractional cutoff (0.1) to produce a normalized and/or internally reported value of 2.5. Alternatively, a DBS measurement of 0.05 (negative) would be corrected to a value of 0.5 (0.05/0.1).

Thus, in an embodiment, the COI is normalized to a serum assay. Thus, in certain embodiments, the COI for a positive sample is ≥1.0 and the COI for a negative sample is <1.0.

Alternatively, in an embodiment, an asymmetric indeterminate region around the cutoff is used to maximize both negative and positive predictive values relative to the predicate method. For example, in certain embodiments, an indeterminate DBS result may range from 0.65 to 2.0 COI where 1.0 represents the negative/positive cutoff. The range of results may be chosen to represent approximately 0.9% of samples that are expected. In this embodiment, patient results that are measured to be within the indeterminate region (DBS COI of 0.65 to 2.0) may be reported as requiring a retest after 7 days and/or testing of a venous blood sample provided by a phlebotomist. Thus, in certain embodiments, the method may comprise reporting a sample as positive when the COI is greater than 2.0, negative when the COI is less than 0.65 and indeterminate when the COI is ≥0.65 COI and ≤2.0 COI. Or, other COIs specific to the analytical technique or extraction procedure may be determined as detailed herein. In one embodiment, there is a defined population that falls in the indeterminate range. For example, the indeterminate range may be defined so as to comprise less than 10%, or less than 5%, or less than 4%, or less than 3%, or less than 2%, or less than 1%, or less than 0.5% of the population at large or the testing population.

At this point, the results may be reported to the subject or their healthcare provider (FIG. 1 (12)). Such results may be used to determine if retesting and/or continued monitoring of the subject is advised.

### Systems for Detection of SARS-CoV-2 Analytes in Dried Samples

Also disclosed are systems for performing any of the steps of the disclosed methods and computer-implemented instructions for performing any of the steps of the disclosed methods or running any of the parts of the disclosed systems.

For example, disclosed is a system comprising one or more stations or components for performing any of the previous method embodiments. In certain embodiments, the system may comprise one or more stations or components for performing any of the steps of: (a) obtaining a dried sample from a subject; (b) extracting the analyte of interest from the dried sample; and (c) detecting the analyte of interest extracted from the dried sample. Also, in certain embodiments, the system may comprise one or more data processors; and a non-transitory computer readable storage medium containing instructions which, when executed on the one or more data processors, cause the one or more data processors to perform processing comprising: (a) obtaining a dried sample from a subject; (b) extracting the analyte of interest from the dried sample; and (c) detecting the analyte of interest extracted from the dried sample.

In certain embodiments, the analyte of interest is an analyte specific to SARS-CoV-2. For example, the SARS-CoV-2 specific analyte may be an antibody to SARS-CoV-2.

A variety of dried samples may be used. In certain embodiments, the dried sample is a dried blood spot. Or, the sample may be dried plasma.

The system may be used to provide a cutoff index (COI) as disclosed in detail herein. For example, in certain embodiments, the system may provide a cutoff index (COI) indicative of whether the subject has a detectable amount of the analyte and so is defined as positive, or does not contain a detected amount of the analyte and so is defined as negative. In certain embodiments, the COI is calculated to incorporate dilution of the sample that occurs during extraction of the SARS-CoV-2 specific analyte from the dried sample. Additionally and/or alternatively, the measured value is divided by a fractional cutoff to account for the dilution so as to provide a normalized COI value. In certain embodiments, the normalized COI comprises a cutoff value for serum. Thus, in certain embodiments, the COI for a positive sample is ≥1.0 and the COI for a negative sample is <1.0. In certain embodiments, the system may be used to further provide a COI range indicative that the sample is not determinate as being either positive or negative for the analyte of interest. For example, in some embodiments, the COI for a positive sample is greater than 2.0, the COI for a negative sample is less than 0.65 and the COI for an indeterminate sample is ≥0.65 COI and ≤2.0 COI.

A variety of techniques may be used to measure the analyte of interest. For example, in one embodiment, wherein the analyte of interest is an antibody, the antibody is measured using a sandwich assay employing a first antigen labeled with a detectable moiety and a second antigen labeled with a binding agent. For example, in certain embodiments, the detectable moiety on the first antigen is an electrochemical moiety. Also, in certain embodiments, the binding agent is streptavidin. For example, as disclosed herein, in certain embodiments, the first antigen:antibody:second antigen complex is bound to a biotin-labeled electrode such that application of a voltage results in a chemiluminescent Emission.

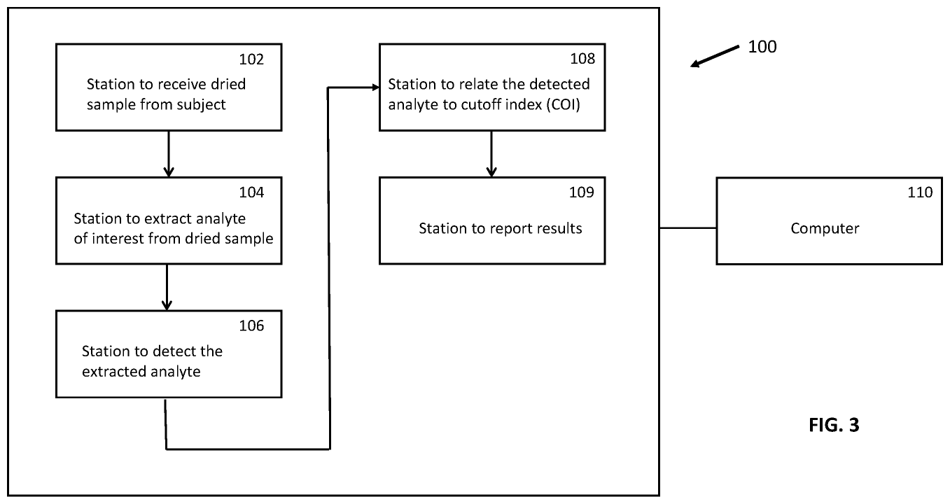

Thus as illustrated in FIG. 3 the system (100) may comprise a station or component for receiving the dried sample (e.g., DBS) (102). The system may further comprise a station or component for extracting the analyte of interest from the dried sample (104). Also, the system may comprise a station or component for detecting the presence of, or measuring the amount of, the analyte (106). Finally, the system may comprise a station or component for relating the detected analyte to defined COIs (108) and a station (or component) to report the results (109).

In some embodiments, the system (100) further comprises a computer (110) and/or a data processor configured to run any of the stations of the system. As disclosed herein, in certain embodiments, the system may comprise one or more computers, and/or a computer product tangibly embodied in a non-transitory computer readable storage medium containing instructions which, when executed on the one or more data processors, cause the one or more data processors to perform actions for performing any of the steps of the methods or implementing the systems or portions of the systems of any of embodiments disclosed herein. One or more embodiments described herein can be implemented using programmatic modules, engines, or components. A programmatic module, engine, or component can include a program, a sub-routine, a portion of a program, or a software component or a hardware component capable of performing one or more stated tasks or functions. As used herein, a module or component can exist on a hardware component independently of other modules or components. Alternatively, a module or component can be a shared element or process of other modules, programs or machines. For example, the system may comprise a computer and/or computer-program product tangibly embodied in a non-transitory machine-readable storage medium for relating the measured analyte values to a COI. Thus, in certain embodiments, the system may comprise components to quantify the measurement. Also, the system may comprise components to perform statistical analysis of the data.

Also disclosed is a computer-program product tangibly embodied in a non-transitory machine-readable storage medium, including instructions configured to cause one or more data processors to perform processing comprising running any of the stations/components of the system and/or performing a step or steps of the methods of any of the disclosed embodiments. In one embodiment, the system comprises a computer-program product tangibly embodied in a non-transitory machine-readable storage medium, including instructions configured to identify the presence of and/or determine the amount of the analyte in the extracted sample. Additionally and/or alternatively, the computer program product may comprise instructions configured to identify the presence of and/or determine the amount of analyte in the original sample. Or, the computer program product may comprise instructions for defining COI values. As noted above, this may depend on the sensitivity of the assay and/or the prevalence of the analyte of interest and/or pathogen from which it is derived in the population.

Embodiments of the disclosed methods and systems have been used to validate a method for detection of antibodies to the severe acute respiratory syndrome coronavirus 2 (SARS-CoV-2) using dried blood spot (DBS) samples with a modified Roche Elecsys Anti-SARS-CoV-2 (ACOV2) immunoassay. In an embodiment, the DBS assay is modified to use an increased aspiration volume (30 μL) compared to the Roche FDA EUA (Emergency Use Authorization) approved Elecsys Anti-SARS-CoV-2 assay with a 12 μL aspiration. This increased aspiration volume accounts, at least in part, for dilution inherent with extraction of DBS samples. Detection of SARS-CoV-2 antibodies in an individual is indicative of previous infection with the virus and the development of an adaptive immune response. Also, in an embodiment, the assay provides a discrete cutoff index (COI) for samples that are positive or negative, and/or positive or negative or indeterminate. Such results can then be reported to the subject providing the DBS sample or their health care provider.

### Examples

### Example 1 Application of Dried Blood Spots to COVED-19 Detection

In one non-limiting embodiment, the methods and systems of the disclosure were used with the Roche Elecsys Anti-SARS-CoV-2 (ACOV2) assay. The Roche Anti-SARS-CoV-2 dried blood spot (DBS) assay is for the detection of total antibodies to SARS-CoV-2 DBS samples from individuals with current or prior COVID-19 infection. The test is intended for use as an aid in identifying individuals with an adaptive immune response to SARS-CoV-2, indicating recent or prior infection. At this time, it is unknown for how long antibodies persist following infection and if the presence of antibodies confers protective immunity. Collection of DBS samples includes assisted (e.g., collected by a trained phlebotomist) as well as self-collection by the patient (e.g., in a home setting). The SARS-CoV-2 DBS assay utilizes an electrochemiluminescence immunoassay “ECLIA” based on the sandwich principle and is intended for use on cobas e immunoassay analyzers. The Elecsys Anti-SARS-CoV-2 assay uses a recombinant protein representing the nucleocapsid (N) antigen for the determination of antibodies against SARS-CoV-2. The assay duration is about 18 minutes.

As noted above, results are for the detection of total SARS-CoV-2 antibodies (without differentiation) from dried blood spot extracts. Antibodies to SARS-CoV-2 are generally detectable in blood several days after initial infection, although the duration of time antibodies are present post-infection is not well characterized. The sensitivity of the Roche Anti-SARS-COV-2 DBS assay is dependent on number of days post contraction of SARS-CoV-2. However, negative results do not preclude acute SARS-CoV-2 infection. If acute infection is suspected, direct testing for SARS-CoV-2 may be necessary. Also, false positive results for the Elecsys Anti-SARS-CoV-2 assay may occur due to cross reactivity from pre-existing antibodies or other possible causes.

This example demonstrates the performance characteristics of dried blood spot cards (specifically Perkin Elmer 226 Spot Saver cards) to ensure they are reliable and suitable for a modified SARS-CoV-2 Roche assay, and can be used for self-collection in a homelike setting. Validation included evaluations of imprecision, carryover, stability (shipping, room temperature, operational, and quality control), clinical agreement (assisted and self-collected DBS as well as contrived samples at the assay cutoff), various robustness studies (effects hemolysis, alcohol, and finger contamination), as well as assessment of an alternate collection card.

Assay Principle

The Roche Anti-SARS-CoV-2 DBS assay was used with the Roche e801 immunoassay module as part of a Roche Cobas 8000 instrument which can measure samples in Hitachi microcups. As the microcups have a listed dead volume of 50 μL compared to 100 μL for the standard sample cup, this reduces the volume requirement for measurement and influenced the volume of extraction buffer that was utilized (see below).

The assay for COVID-19 antibodies is based on the sandwich principle. During the first incubation, 30 μL of sample (increased from 12 μL for the EUA approved Roche Anti-SARS-CoV-2 assay), a biotinylated SARS-CoV-2-specific recombinant antigen and SARS-CoV-2-specific recombinant antigen labeled with a ruthenium complex were mixed to form a sandwich complex. After addition of streptavidin-coated microparticles, the complex became bound to the solid phase via interaction of biotin and streptavidin. The reaction mixture was aspirated into the measuring cell where the microparticles were magnetically captured onto the surface of the electrode. Unbound substances were then removed and application of a voltage to the electrode then induced chemiluminescent emission which was measured by a photomultiplier.

The Roche Anti-SARS-CoV DBS assay uses the Roche Elecsys Anti-SARS-CoV-2 assay kit (Roche Diagnostics/Indianapolis, Ind., Elecsys Anti-SARS-CoV-2 Application Sheet, Reagent Catalog No. 09203079190) containing both reagent and calibrator. Reagents and calibrators are used as received and were not modified for use.

Sample Extraction and Measurement

Dried blood spot samples were acquired by lancing a patient's finger (e.g. BD Microtainer Contact-Activated Blue Lancet, Blade: 1.5 mm wide×2.0 mm deep) following disinfection with an alcohol wipe. Following wiping away the initial drop of blood with gauze, the patient applied blood to the pre-defined locations on the DBS card. The pre-defined locations were indicated by five dashed circles; each circle was approximately ½ inch in diameter. The collection process can be performed as a self-collection or as an assisted collection.

Following collection, the sample was allowed to dry completely for 3 hours at room temperature. The DBS card was then folded and placed in a plastic biohazard bag used for sample return. Once received in the laboratory, the samples were reviewed for collection quality (number and diameter of blood spots). Samples were extracted by making two (2) blood saturated ¼″ diameter round punches from each patients' DBS card. If 2 blood saturated punches were unable to be made, the sample was rejected for analysis.

Punches were placed in a 16×75 mm polypropylene tube with 150 μL extraction buffer (Roche MultiAssay diluent). After punches were submerged in buffer, samples tubes were covered with parafilm and placed on an orbital mixer for 1 hour at room temperature. Following extraction, the punches were removed and squeezed to remove any remnant liquid. The extracts were then transferred to a Hitachi microcup for measurement using the Roche Anti-SARS-CoV-2 DBS assay on a Roche e801.

Although 150 μL was used for extraction, the volume recovered during the extraction process was close to 100 μL due to absorption and adsorption. This volume was selected to maximize assay sensitivity while also allowing consistent measurement on the autoanlyzer with microcups.

### Example 2—Assay Calibration

Determination of DBS Cutoff

For the FDA EUA approved SARS-CoV-2 Roche serum assay, positive and negative results were determined automatically with measurements producing a numerical result relative to the assay's negative/positive cutoff index (COI). Values less than 1.0 were reported as negative and measurements having a COI greater than or equal to 1.0 were reported as positive. To determine the cutoff for DBS samples, the assay was initially calibrated using the neat calibrator materials (un-modified) that were included the Anti-SARS-CoV-2 reagent kit. However, due to dilution through extraction of DBS samples, a relative cutoff value was defined in order to determine negative and positive results.

To set the cutoff for negative and positive DBS samples, the neat calibrator materials (two levels at 0.080 and 1.233 COI) were spotted onto DBS cards and extracted in replicates of two over 13 batches. Using the results from the neat extracted calibrator, the cutoff for DBS samples was assigned by dividing the extracted results (mean measured calibrator DBS extract COI of 0.0817) by the neat calibrator results (mean measured COI of 1.233) to obtain a relative DBS cutoff value COI of 0.0666.

To set the cutoff for negative and positive DBS samples, the neat calibrator materials (two levels at 0.080 and 1.233 COI) were spotted onto DBS cards and extracted in replicates of two over 13 batches. Using the results from the neat extracted calibrator, the cutoff for DBS samples was assigned by dividing the extracted results (mean measured calibrator DBS extract COI of 0.0817) by the neat calibrator results (mean measured COI of 1.233) to obtain a relative DBS cutoff value COI of 0.0666.

Quality Control Testing and Interpretation of Results

To perform quality control (QC) testing, negative and positive control materials were added to DBS cards by pipetting 40-50 μL of the neat (undiluted) material onto each spot required. Dried QCs were allowed to dry for 3 hours at room temperature prior to storage until use. Extraction of the materials required punches to be saturated with the QC material (and not blood). Successful measurements of each QC extract occurred on every reagent pack utilized prior to measurement of patient samples. For validation studies, Technopath MultiChem Covid-19 negative and positive quality controls were utilized. Extracted QC materials were expected to have negative (COI <1.0) and positive (COI >1.0) results following normalization to the cutoff.

All valid measured results were divided by the relative DBS assay cutoff COI of 0.0666. This step was performed to normalize the results such that measurements greater than or equal to 1 from DBS indicated a SARS-CoV-2 antibody positive status and results less than 1 indicated a negative status, similar to the Roche EUA approved Elecsys Anti-SARS-CoV-2 assay.

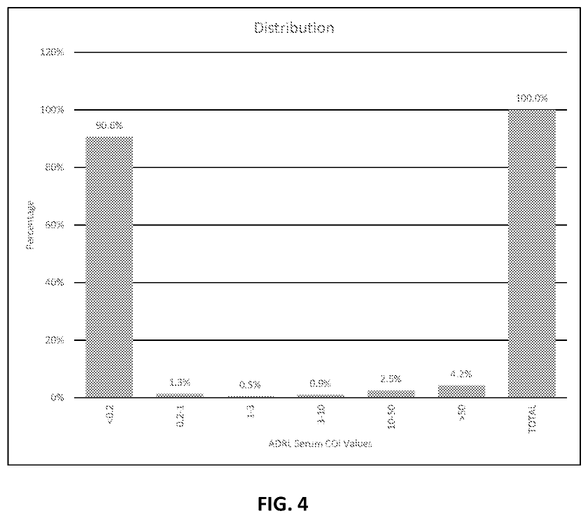

An asymmetric indeterminate region around the cutoff was established to maximize both negative and positive predictive values relative to the predicate method. Indeterminate DBS results ranged from 0.65 to 2.0 COI where 1.0 represented the negative/positive cutoff in the data. This range was approximately equal to a serum range of 0.46 to 2.71 COI which represents approximately 0.9% of samples that were expected (FIG. 4)

Considerations for Validation Studies

Due to the difficulty in obtaining fingerstick DBS samples from seropositive donors with a range of COI values, contrived blood samples were utilized for the majority of the studies described below, with a particular focus on creating samples within 5 fold of the serum cutoff. Contrived samples were created by slowly mixing serum with blood cells (plasma removed) obtained from EDTA blood from a seronegative donor with Type 0 blood to create contrived samples with a hematocrit of 40%. Both negative and serum SARS-CoV-2 antibody positive samples (as determined using the Roche Anti-SARS-CoV-2 assay) were utilized.

After creation of the contrived blood samples, 40-50 μL of contrived blood was dosed onto Perkin Elmer 226 Spot Saver (or Whatman and Eastern Business Forms 903) cards to create each DBS sample. Samples were dried for 3 hours at room temperature prior to extraction or storage.

Additionally, measurement of neat (undiluted) serum samples was performed using an e602 module of a Roche Cobas 8000 instrument with the EUA approved Roche Elecsys Anti-SARS-CoV-2 assay for comparison purposes.

Distribution of Serum Results

In order to gain a better understanding of the distribution of expected DBS samples, serum results were monitored over 8 days using the EUA approved Roche Elecsys Anti-SARS-CoV-2 assay. The distribution of 13,057 measurements of patient serum results were generally less than 0.2 COI for negative serum results (90.6% of all results) or greater than 3.0 COI for positive serum results (7.6% of all results) (FIG. 4). The remaining 1.8% of results were observed within the COI range of 0.2 to 3.0. These results and the distribution observed were used to inform study construct (i.e. selection of high negative samples and low positive samples).

### Example 3—Performance Evaluation

Cutoff Verification Study

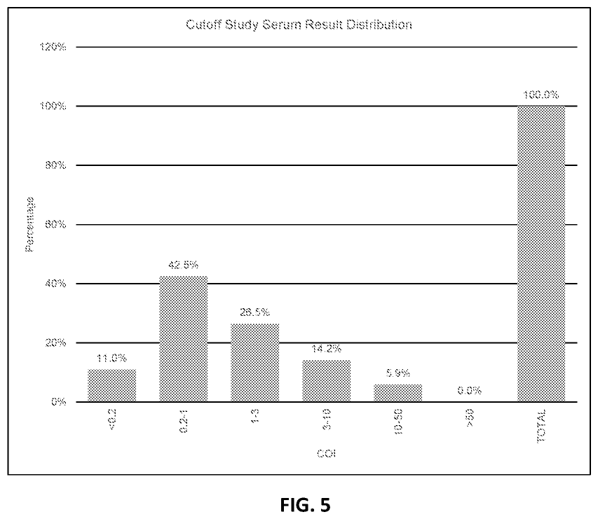

To test the performance of the assay near the serum cutoff index, 220 serum samples were acquired with 69% of the serum measurements from these samples ranging from 0.2 to 3.0 COI (FIG. 5). It is important to note that the difference in distribution of the serum results used for this study (69% of serum results within 0.2 to 3.0 COI) compared to the frequency of serum samples expected within this range (1.8% of serum results within 0.2 to 3.0 COI).

Contrived blood samples from these serum samples were spotted, extracted, and measured as described above. The results were compared to serum measurements from the same samples measured in parallel using the EUA approved serum assay as described above for Distribution of Serum Results. Comparison was performed using the Qualitative Method Comparison module in EP Evaluator as well as linear regression in Excel.

Additionally, the measured results were analyzed in the context of distribution of expected results. This approach considers less than 100% agreement near the serum COI cutoff, together with 100% agreement for “true” negative (serum COI <0.2) and “true” positive (serum COI >3.0) results. Serum specimens with COI <0.2 and >3 account for 98.2% of samples expected (FIG. 4).

Distribution of serum samples utilized for the cutoff verification study can be observed in FIG. 5. Comparing the distribution of the samples utilized to the expected distribution of samples (FIG. 4), a larger percentage (69.0%) of samples near the cutoff (COIs of 0.2 to 3.0) were utilized as compared to the percentage (1.8%) of actual patient samples in this range.

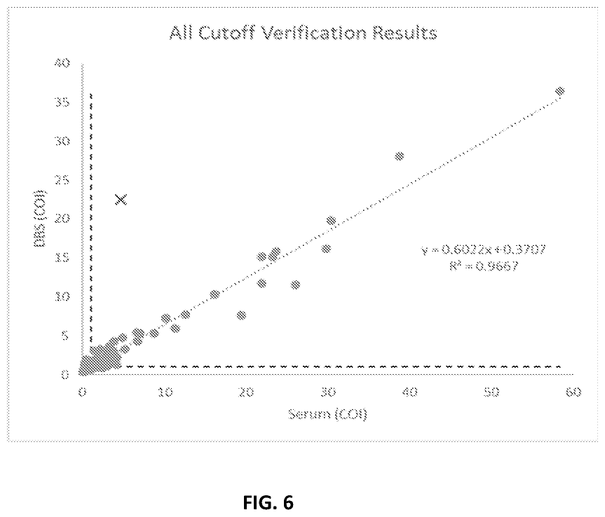

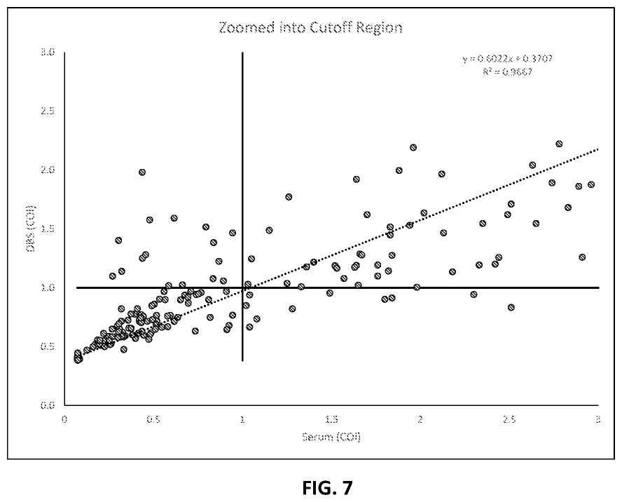

The categorical agreement of these results is summarized in Table 1. The total agreement of categorical results was 87.7% with a sensitivity of 89.3% and a specificity of 86.3%. However, results were also found to be correlated (R²>0.96) over the range of results tested (FIGS. 6 and 7). The dashed lines in the graphs indicate the negative and positive cutoff index of 1 for both serum and DBS results where the measured DBS results are normalized to the true cutoff value. One sample (shown as X in FIG. 6) was deemed to be an outlier (EP Evaluator) and excluded from the analysis.

TABLE 1
Summary of Categorical Agreement of Results

| | Negative Reference | Positive Reference | Total |
|---|---|---|---|
| Negative Test | 101 | 11 | 112 |
| Positive Test | 16 | 92 | 108 |
| Total | 117 | 103 | 220 |

The correlation of the results (FIGS. 6 and 7) as well as the distribution of expected results (FIG. 4) were used to analyze the data in the context of the expected distribution. These results were combined to generate observed agreements for specific sample ranges as shown in Table 2. Complete (100%) categorical agreement was expected for samples with COI results less than 0.2 and greater than 3.0 and 82.8% agreement was separately observed for samples with COIs ranging from 0.2 to 1.0 and from 1.0 to 3.0. The observed agreements for these individual ranges (Table 2) were utilized to project agreements for 13,056 patient samples (Table 3) based on the frequency of results within these regions.

TABLE 2
Agreement of Expected Results

| Serum COI | Frequency of | Observed |
|---|---|---|
| Range | Results (%) | Agreement (%) |
| <0.2 | 90.6 | 100 |
| 0.2-1.0 | 1.3 | 82.8 |
| 1.0-3.0 | 0.5 | 82.8 |
| >3.0 | 7.6 | 100.0 |
| Total | 100.0 | |

As expected due to the low prevalence of samples with serum COIs ranging from 0.2 to 3.0, the total agreement for the expected distribution of patient samples was 99.7% with a sensitivity of 99.0%%, a specificity of 99.8%, a positive predictive value of 97.6%, and a negative predictive value of 99.9% (Table 3).

TABLE 3
Summary of Categorical Agreement of Results

| | Negative Reference | Positive Reference | Total |
|---|---|---|---|
| Negative Test | 11970 | 11 | 11981 |
| Positive Test | 29 | 1046 | 1075 |
| Total | 11999 | 1057 | 13056 |

The results from this study were also used to set the indeterminate range. The highest false positive observed had a DBS COI value of 1.98 and the lowest false negative observed had a DBS COI value of 0.667. Based on these results, the DBS indeterminate range for patient samples was set from 0.65 to 2.0 COI.

Intra-Assay Imprecision Study

Intra-assay imprecision measurements were performed using two different study constructs. In the first construct, 20 replicates of the same contrived blood sample were spotted onto DBS cards, extracted, and analyzed individually to assess the imprecision of the entire process. This was performed with 5 different levels of antibody including 2 negative and 3 positive contrived blood samples.

Additionally, 20 replicates of contrived blood samples were spotted onto DBS cards, extracted but then extracts were pooled and mixed prior to repeat measurement from the post-extraction pooled DBS extracts to assess the imprecision of the measurement system, independent of the imprecision of the DBS extraction process. The second study was performed using 1 negative and 1 positive contrived blood samples.

Intra-assay imprecision measurements investigating the precision of the entire process yielded 100% categorical agreement for 4 of the 5 whole blood samples tested. Whole blood sample 2 demonstrated 95% categorical agreement and was within 10% of the DBS COI cutoff (0.911, 5.3% CV). Intra-assay imprecision evaluation of the measurement process yielded 100% categorical agreement for the positive sample tested with a mean DBS COI of 3.54 and 90% agreement for the negative sample tested. The negative sample had a mean DBS COI within 4% of the cutoff (0.966) and an observed imprecision of 2.2%.

For samples within 15% of the cutoff, a high level of precision was observed (CV <6% for each sample) but samples did not always display 100% categorical agreement. This data suggests that an indeterminate region may be necessary for samples that measure close to the cutoff index.

Inter-Assay Imprecision Study

Inter-assay imprecision was performed over 4 different batches (5 replicates/batch) with 20 replicates of 2 levels of QC materials and 4 contrived blood samples covering a range of negative and positive COI values. Once data was obtained, the average value (mean), standard deviation (SD), and coefficient of variation (CV) for inter-assay imprecision was determined as well as the overall agreement.

Inter-assay imprecision measurements indicated a total agreement of 100% for the 4 contrived blood DBS samples over the course of the study. Extracted QC results had a total agreement of 95% or greater over the course of the study.

Puncher Carryover Study

Low and high level contrived DBS whole blood patient samples were assayed following spotting and extraction for evaluation of punch carryover. The following punching order was utilized for the analysis of 10 replicates of the High pool (H) and 11 replicates of the Low (L) pool for evaluation in the EP Evaluator Carryover Module: L L L H H L H H L L L L H H L H H L H H L. The low and high samples had mean DBS COIs of 0.42 and 159.4, respectively. The low-low result was a low result that immediately followed another low result, while a high-low result was a low result that immediately followed a high result. Carryover was defined as the mean of the high-low results minus the mean of the low-low results. The error limit was calculated as three times the SD of the low-low results. The carryover test passed if the calculated carryover was less than the error limit. The results showed the carryover to be less than three times the standard deviation of the low-low results.

Hemolyzed Sample Study

To test the effects of hemolysis, five positive and five negative contrived whole blood samples were created and duplicate paired aliquots made for a split pairwise evaluation of the impact of hemolysis. One paired aliquot for each contrived sample (5 positive, 5 negative) was flash frozen at −70° C. for 30 minutes and thawed to hemolyze the red blood cells. Both aliquots from each donor (lysed and un-lysed) were then spotted simultaneously. Following recommended drying, the samples were extracted and assayed within the same run.

Results from hemolyzed sample pairs were compared to un-hemolyzed samples. Unlysed and lysed blood samples showed 100% agreement for the 10 samples measured (5 negative and 5 positive).

Clinical Agreement Through Phlebotomy Assisted Collection Study

At least 30 negative donors provided venous serum, DBS samples from a lanced finger with phlebotomy assistance, and a nasal swab for PCR testing. Additionally, more than 30 previously PCR confirmed positive individuals provided venous serum samples and phlebotomy assisted DBS samples from a lanced finger. Comparative results between serum and assisted collection DBS were analyzed using the Qualitative Method Comparison module in EP Evaluator.

In total, 99 DBS samples were measured after phlebotomy assisted collection for comparison of PCR status versus DBS results. Fifteen of the reported PCR positive donors were repeat donors (i.e. the same patient provided 2 samples at different times). Comparing the observed DBS results to reported SARS-CoV-2 PCR status indicated an overall agreement of 98.0%, sensitivity of 96.6% and specificity of 100.0%. Negative and positive predictive values were 95.2 and 100.0%, respectively (Table 4).

TABLE 4
Assisted Collection PCR

| | Negative Reference | Positive Reference | Total |
|---|---|---|---|
| Negative Test | 40 | 2 | 42 |
| Positive Test | — | 57 | 57 |
| Total | 40 | 59 | 99 |

Comparison of paired serum and assisted collection DBS samples indicated overall agreement of 100%, sensitivity of 100% and specificity of 100.0%. Negative and positive predictive values were both 100.0% (Table 5).

TABLE 5
Assisted Collection Serum

| | Negative Reference | Positive Reference | Total |
|---|---|---|---|
| Negative Test | 42 | — | 42 |
| Positive Test | — | 56 | 56 |
| Total | 42 | 56 | 98 |

Results again included duplicate samples from 14 individuals. All measurements from duplicate donors were in categorical agreement, i.e. no changes were observed over time (Table 6). Additionally, negative predictive agreement for assisted collection was 100% (Table 7).

TABLE 6
Time-Dependence of Positive Results for Assisted Collection

| | | Candidate Device Results | | |
|---|---|---|---|---|
| | | Total | | |
| Days from | Number of | Antibody | Total | |
| Symptom | Samples | Positive | Antibody PPA | |
| Onset | Tested | Results | (%) | 95% CI |
| 0-7 days | 0 | 0 | NA | NA |
| 8-14 days | 0 | 0 | NA | NA |
| ≥15 days | 99 | 97* | 98.0% | 92.9%-99.7% |

*The two false negatives observed were duplicate samples from the same donor.

TABLE 7
Negative Predictive Agreement Observed for Assisted Collection

| | |
|---|---|
| Number of | Candidate Device Results |

| Samples | Negative | Total Antibody |
|---|---|---|
| Tested | Results | NPA (%) |
| 40 | 40 | 100.0% |

Clinical Agreement Through Self-Collection Study

An additional clinical agreement study was performed with a similar construct to the study described above. At least 30 negative and 30 positive DBS collection naïve donors without laboratory or sample collection experience were provided with materials and instructions (i.e. a self-collection kit) and asked to follow instruction to acquire a DBS sample via fingerstick. The collection process took place at home or in a home-like environment where participants were provided a kit including instructions for use, sample collection tools and a pre-paid return label. Donors were observed in person during the self-collection process. Additionally, donors provided serum samples and if not previously confirmed to be COVID 19 positive, a nasal swab for PCR testing. Results were analyzed using the Qualitative Method Comparison module in EP Evaluator.

Qualitative comparison of 70 successfully self-collected DBS samples (39 donors PCR negative and 31 donors PCR positive) yielded a total agreement of 98.6% with a sensitivity of 96.8% and a specificity of 100.0% (Table 8). Additionally, the negative and positive predictive values were found to be 97.5 and 100.0%, respectively. Comparison of paired serum and self-collected DBS samples indicated overall agreement of 100%, sensitivity of 100% and specificity of 100.0% (Table 9) with no changes observed over time (Table 10). Additionally, negative predictive agreement for assisted collection was 100% (Table 11). Negative and positive predictive values were both 100.0%.

TABLE 8
Self-Collection PCR

| | Negative Reference | Positive Reference | Total |
|---|---|---|---|
| Negative Test | 39 | 1 | 40 |
| Positive Test | — | 30 | 30 |
| Total | 39 | 31 | 70 |

TABLE 9
Self-Collection Serum

| | Negative Reference | Positive Reference | Total |
|---|---|---|---|
| Negative Test | 40 | — | 40 |
| Positive Test | — | 28 | 28 |
| Total | 40 | 28 | 68 |

TABLE 10
Time-Dependence of Positive Results for Self-Collection

| | | Candidate Device Results | | |
|---|---|---|---|---|
| | | Total | | |
| Days from | Number of | Antibody | Total | |
| Symptom | Samples | Positive | Antibody PPA | |
| Onset | Tested | Results | (%) | 95% CI |
| 0-7 days | 0 | 0 | NA | NA |
| 8-14 days | 0 | 0 | NA | NA |
| ≥15 days | 31 | 30 | 96.8% | 83.8%-99.4% |

TABLE 11
Negative Predictive Agreement Observed for Self-Collection

| | |
|---|---|
| Number of | Candidate Device Results |

| Samples | Negative | Total Antibody |
|---|---|---|
| Tested | Results | NPA (%) |
| 39 | 39 | 100.0% |

Shipping Stability (Temperature Excursion) Study

Forty serum samples were used to create contrived blood DBS samples that were subjected to summer and winter temperature excursion tests for shipping stability purposes. Each of the 40 samples was spotted in triplicate with one set being used for baseline measurements (stored at room temperature), one set being subjected to summer temperature excursion, and one set being subjected to a winter temperature excursion (Table 12). Samples utilized for baseline measurements were stored at room temperature throughout the study and measured during the same batch as samples subjected to temperature excursions. Excursion results were compared to baseline measurements to determine if interpretation changed as a result of the excursion.

For the winter shipping study, 38 of 40 samples (95%) were in qualitative agreement with baseline measurements. For the summer shipping study, 37 of 40 samples (92.5%) were in qualitative agreement with baseline measurements. Discordant shipping excursion samples (summer or winter) had baseline results ranging from a DBS COI of 0.937 to 1.28.

TABLE 12
Temperature Excursion Study Procedure

| Cycle | Winter | Summer | |
|---|---|---|---|
| Period | Temperatures* | Temperatures* | Hours |
| 1 | 22° C. drying | | 3 |
| 2 | 22° C. in plastic bag | | 5 |
| 3 | −20° C. | 40° C. | 8 |
| 4 | 22° C. | 22° C. | 4 |
| 5 | −20° C. | 40° C. | 2 |
| 6 | 4° C. | 30° C. | 36 |
| 7 | −20° C. | 40° C. | 6 |

*Within ±2° C.

Room Temperature Stability Study

Negative and positive contrived blood samples were spotted onto DBS cards and assayed in triplicate for each time point. All samples were stored overnight at room temperature. Baseline measurements (Day 0) were made on the day following sample creation after drying for 3 hours at room temperature and storage overnight in a bag. Additional measurements were made on Days 7, 15, and 35 after storage at room temperature (20-24° C.). Contrived blood samples utilized had DBS COI values ranging from negative to moderately positive. Room temperature stability results demonstrated 100% agreement when stored for up to 35 days.

Operational Stability Study

Post extraction stability was assessed using a total of 48 DBS extracts ranging from a DBS COI of 0.381 to 22.5. Following initial measurement, samples were parafilmed and stored at room temperature for 24 hours prior to re-measurement.

Of the 45 samples measured, 43 (95.6%) of the re-measured results demonstrated categorical agreement with baseline measurements.

Quality Control Stability Study

Quality control material stability was assessed using two levels of QC materials spotted directly onto Perkin Elmer 226 cards (added neat, not as a contrived blood). QC spotted DBS cards were allowed to dry for 3 hours prior to storage in bags at room temperature. Each QC level was then measured in triplicate for baseline measurements (on the day following creation) and for up to 15 days to ascertain QC storage stability for operational use in QC monitoring. DBS spotted neat QC material stability was demonstrated to be in 100% categorical agreement with baseline measurements on both Day 7 and Day 15 of measurements.

Alternative Drying Time Study

Although the recommended room temperature drying time for samples prior to storage and shipment is 3 hours, alternative drying times were studied in the event a donor does not follow the instructions for use (IFUs). Five positive and five negative contrived blood DBS samples were spotted in quadruplicate. The dried blood spot samples were allowed to dry for different lengths of time at room temperature prior to placing in the return bags for the following times: immediately, 1 hour, 3 hours (recommended), and overnight (20.5 hours). Samples were then extracted on the following day, assayed in a single run and results compared to the recommended drying condition.

For the 10 samples utilized for this study, 100% categorical agreement was observed across the three drying time points evaluated (up to 20.5 hours) when compared to baseline measurements.

Effects of Elevated Humidity and Temperature during Drying Study

Five positive and five negative contrived blood DBS samples were spotted in quadruplicate. One of the four samples was allowed to dry using the recommended procedure, 3 hours at room temperature (<50% relative humidity), prior to placing in the return bag. This sample was used to determine baseline results.

To study the effects of drying under a relative humidity greater than 80%, the three remaining dried blood spots from each donor were then placed in an incubator set to 30° C. with a water pan in the bottom immediately following spotting (and prior to placing in a return bag). Samples were left in the incubator for the following time points: 1 hour, 3 hours, and overnight (20.5 hours). After removal, the samples were placed in return storage bags until measurement. Samples were extracted on the following day, assayed in a single run and results compared to the recommended drying condition.

For the 10 samples utilized for this study, 100% categorical agreement was observed across the three time points and >80% relative humidity when compared to baseline measurements.

Effects of Alcohol Addition to DBS card Study

Five positive and five negative contrived blood DBS samples were created. Prior to spotting, one DBS spotting region for each sample was exposed to alcohol by wiping an alcohol pad across the spot location immediately (<2 seconds) prior to spotting. Samples were then spotted onto the alcohol exposed spots and “clean” spots. Following recommended drying (3 hours at room temperature) and overnight storage at room temperature, samples were extracted on the following day, assayed in a single run and results from alcohol exposed spots were compared to samples from “clean” spots.

Dried blood spot cards exposed to alcohol, mimicking donor contamination through improper collection, demonstrated 100% categorical agreement for the 10 samples utilized for this study.

Effects of Finger Contamination Study

Five positive and five negative contrived blood DBS samples were created. Prior to spotting, one DBS spotting region for each sample was “contaminated” by pressing an ungloved finger directly to the spot location. Samples were then spotted onto the contaminated spot locations and “clean” spots. Following recommended drying (3 hours prior to bagging) and overnight storage at room temperature, samples were extracted on the following day, assayed within a single run and results from contaminated spot locations were compared to samples from “clean” spots.

Dried blood spot cards exposed to contamination, mimicking a donor contaminating the collection card demonstrated 100% categorical agreement for the 10 samples utilized for this study.

Matrix Equivalency—Alternate DBS Card Study (EBF)

Eastern Business Forms (EBF) 903 blood spot cards were investigated as an alternate DBS card by testing equivalence to the Perkin Elmer 226 Spot Saver Card utilized throughout validation. Split pairwise testing was performed with 40 contrived blood DBS samples that were spotted onto both the Eastern Business Forms and Perkin Elmer DBS cards.

A total of 38 of 40 (95%) contrived blood DBS samples were in categorical agreement when comparing Eastern Business Forms 903 DBS cards to Perkin Elmer 226 Spot Saver Cards.

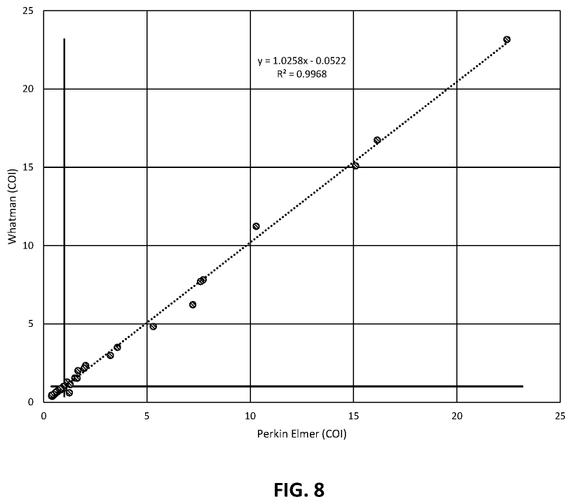

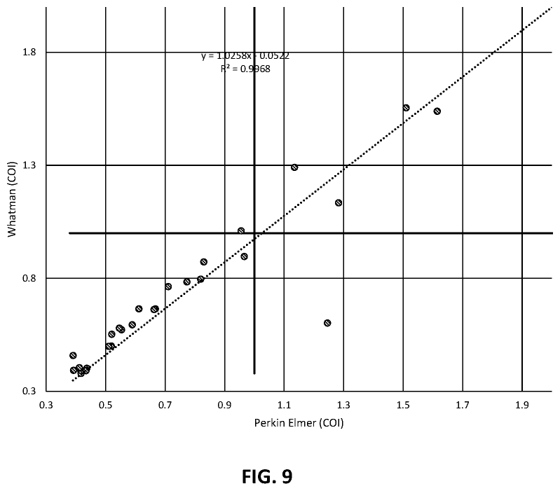

Matrix Equivalency—Alternate DBS Card Study (Whatman) Whatman blood spot cards were investigated as an alternate DBS card by testing equivalence to the Perkin Elmer 226 Spot Saver Card utilized throughout validation. Results show total agreement of 95.0% with strong correlation (R²>0.99) (FIGS. 8 and 9).

Quantitative DBS Feasibility Studies Using an e602 Module of a Roche Cobas 800 Instrument

To test the performance of DBS using an e602 module of a Roche Cobas 800 instrument with the EUA approved Roche Elecsys Anti-SARS-Co-V-2 assay, 40 contrived DBS samples were compared to 40 neat (undiluted) serum samples (n=80 samples).

The contrived blood samples were made with 40% hematocrit following the steps outlined above. After creation of the contrived blood samples, 40-50 μL of contrived blood was dosed onto Perkin Elmer 226 Spot Saver (or Whatman and Eastern Business Forms 903) cards to create each DBS sample. Samples were dried for 3 hours at room temperature prior to extraction or storage.

The following modifications were made to accommodate measurements on e602 module: no microcups were used, DBS samples were made and extracted in duplicate using a Roche Universal diluent as extraction buffer, and 20 μL aspirations were used instead of 12 μL used in the e801.

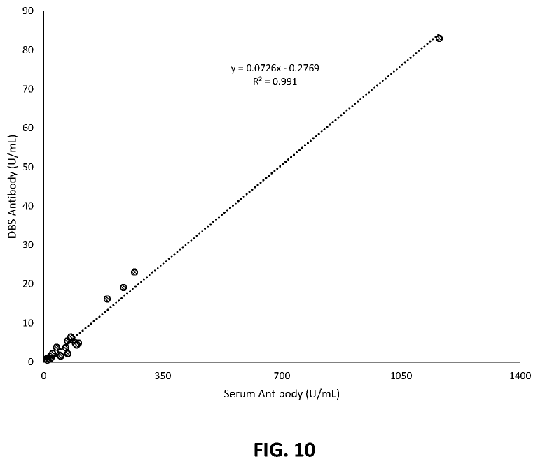

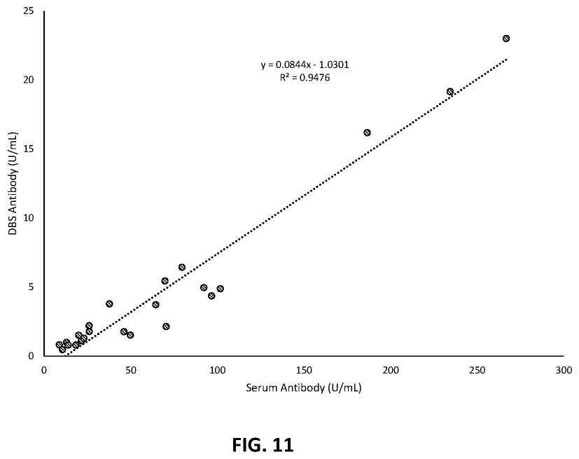

A linear regression line comparing Serum antibodies and DBS antibodies showed overall strong correlation (R²>0.95), and lowest possible measurement of antibody from DBS was ˜8 U/mL, which is 10× greater than cutoff value with current assay (0.8 U/mL) (FIGS. 10 and 11).

### Example 5—Testing of Potential Limitations of Procedure

The liquid assay has no biotin interference in serum concentrations up to 1200 ng/mL. Some studies have shown that serum concentrations of biotin can reach up to 355 ng/mL within the first hour after biotin ingestion for subjects consuming supplements of 20 mg biotin per day (Directive 2000/54/EC of the European Parliament and Council of 18 Sep. 2000 on the protection of workers from risks related to exposure to biological agents at work) and up to 1160 ng/mL for subjects after a single dose of 300 mg biotin (Grimsey P, Frey N, Bendig G, et al. Population pharmacokinetics of exogenous biotin and the relationship between biotin serum levels and in vitro immunoassay interference. Int J Pharmacokinet 2017; 2:247-256, Future Science Ltd London, UK. cited 2018 Jan. 1. Available on the web at: www.futurescience.com/doi/10.4155/ipk-2017-0013). Potential endogenous interferences other than hemolysis and biotin have not been tested and an interference cannot be excluded.

No false negative results due to high-dose hook effect were found with the Elecsys Anti-SARS-CoV-2 liquid assay but occurrence of high-dose hook effect cannot be completely excluded.

SARS-CoV-2 IgG antibodies may be below detectable levels in patients who have been exhibiting symptoms for less than 15 days.

Cross-Reactivity: A total of 10,453 samples were tested (including 792 potentially cross-reacting samples) with the Elecsys Anti-SARS-CoV-2 liquid assay. All samples were obtained before December 2019. Twenty-one (21) false positive samples were detected.

In rare cases, interference due to extremely high titers of antibodies to analyte-specific antibodies, streptavidin or ruthenium can occur. These effects are minimized by suitable test design.

For diagnostic purposes, the results are assessed in conjunction with the patient's medical history, clinical examination and other findings. A negative test result does not rule out the possibility of an infection with SARS-CoV-2. Serum or plasma samples from the early (pre-seroconversion) phase of illness can yield negative findings. Also, over time, titers may decline and eventually become negative.

### Example 6—Embodiments

The disclosure may be better understood by reference to the following non-limiting embodiments.

A1. A method for measuring an analyte of interest in a dried sample comprising: (a) obtaining a dried sample from a subject; (b) extracting the analyte of interest from the dried sample; and (c) detecting the analyte of interest extracted from the dried sample.
A2. A method of any one of the previous or subsequent method embodiments, wherein the analyte of interest is an analyte specific to SARS-CoV-2.
A3. A method of any one of the previous or subsequent method embodiments, wherein the SARS-CoV-2 specific analyte is an antibody to SARS-CoV-2.
A4. A method of any one of the previous or subsequent method embodiments, wherein the dried sample is a dried blood spot.
A5. A method of any one of the previous or subsequent method embodiments, wherein the dried sample is dried plasma.
A6. A method of any one of the previous or subsequent method embodiments, further comprising providing a cutoff index (COI) indicative of whether the subject has a detectable amount of the analyte and so is defined as positive, or does not contain a detected amount of the analyte and so is defined as negative.
A7. A method of any one of the previous or subsequent method embodiments, wherein the COI is calculated to incorporate dilution of the sample that occurs during extraction of the SARS-CoV-2 specific analyte from the dried sample.
A8. A method of any one of the previous or subsequent method embodiments, wherein the measured value is divided by a fractional cutoff to account for the dilution so as to provide a normalized COI value.
A9. A method of any one of the previous or subsequent method embodiments, wherein the normalized COI comprises a cutoff value for serum.
A10. A method of any one of the previous or subsequent method embodiments, wherein the COI for a positive sample is ≥1.0 and the COI for a negative sample is <1.0.
A11. A method of any one of the previous or subsequent method embodiments, further comprising providing a COI range indicative that the sample is not determinate as being either positive or negative for the analyte of interest.
A12. A method of any one of the previous or subsequent method embodiments, wherein the COI for a positive sample is greater than 2.0, the COI for a negative sample is less than 0.65 and the COI for an indeterminate sample is ≥0.65 COI and ≤2.0 COI.
A13. A method of any one of the previous or subsequent method embodiments, wherein the analyte of interest is an antibody, and the antibody is measured using a sandwich assay employing a first antigen labeled with a detectable moiety and a second antigen labeled with a binding agent.
A14. A method of any one of the previous or subsequent method embodiments, wherein the detectable moiety on the first antigen is an electrochemical moiety,
A15. A method of any one of the previous or subsequent method embodiments, wherein the binding agent is streptavidin.
A16. A method of any one of the previous or subsequent method embodiments, wherein the first antigen:antibody:second antigen complex is bound to a biotin-labeled electrode such that application of a voltage results in a chemiluminescent emission.
B1. A system comprising one or more stations or components for performing any of the previous method embodiments.
B2. A system comprising one or more stations or components for performing any of the steps of: (a) obtaining a dried sample from a subject; (b) extracting the analyte of interest from the dried sample; and (c) detecting the analyte of interest extracted from the dried sample.
B3. A system of any one of the previous or subsequent system embodiments, further comprising one or more data processors; and

a non-transitory computer readable storage medium containing instructions which, when executed on the one or more data processors, cause the one or more data processors to perform processing comprising: (a) obtaining a dried sample from a subject; (b) extracting the analyte of interest from the dried sample; and (c) detecting the analyte of interest extracted from the dried sample.

B4. A system of any one of the previous or subsequent system embodiments, wherein the analyte of interest is an analyte specific to SARS-CoV-2.
B5. A system of any one of the previous or subsequent system embodiments, wherein the SARS-CoV-2 specific analyte is an antibody to SARS-CoV-2.
B6. A system of any one of the previous or subsequent system embodiments, wherein the dried sample is a dried blood spot.
B7. A system of any one of the previous or subsequent system embodiments, wherein the dried sample is dried plasma.
B8. A system of any one of the previous or subsequent system embodiments, further comprising providing a cutoff index (COI) indicative of whether the subject has a detectable amount of the analyte and so is defined as positive, or does not contain a detected amount of the analyte and so is defined as negative.
B9. A system of any one of the previous or subsequent system embodiments, wherein the COI is calculated to incorporate dilution of the sample that occurs during extraction of the SARS-CoV-2 specific analyte from the dried sample.
B10. A system of any one of the previous or subsequent system embodiments, wherein the measured value is divided by a fractional cutoff to account for the dilution so as to provide a normalized COI value.
B11. A system of any one of the previous or subsequent system embodiments, wherein the normalized COI comprises a cutoff value for serum.
B12. A system of any one of the previous or subsequent system embodiments, wherein the COI for a positive sample is ≥1.0 and the COI for a negative sample is <1.0.
B13. A system of any one of the previous or subsequent system embodiments, further comprising providing a COI range indicative that the sample is not determinate as being either positive or negative for the analyte of interest.
B14. A system of any one of the previous or subsequent system embodiments, wherein the COI for a positive sample is greater than 2.0, the COI for a negative sample is less than 0.65 and the COI for an indeterminate sample is ≥0.65 COI and ≤2.0 COI.
B15. A system of any one of the previous or subsequent system embodiments, wherein the analyte of interest is an antibody, and the antibody is measured using a sandwich assay employing a first antigen labeled with a detectable moiety and a second antigen labeled with a binding agent.
B16. A system of any one of the previous or subsequent system embodiments, wherein the detectable moiety on the first antigen is an electrochemical moiety,
B17. A system of any one of the previous or subsequent system embodiments, wherein the binding agent is streptavidin.
B18. A system of any one of the previous or subsequent system embodiments, wherein the first antigen:antibody:second antigen complex is bound to a biotin-labeled electrode such that application of a voltage results in a chemiluminescent emission.
C1. A computer-program product tangibly embodied in a non-transitory machine-readable storage medium, including instructions which, when executed on the one or more data processors, cause the one or more data processors to run any part of the previous or subsequent system embodiments.
D1. A system comprising:

one or more data processors; and

a non-transitory computer readable storage medium containing instructions which, when executed on the one or more data processors, cause the one or more data processors to perform processing to perform any of the previous or subsequent system embodiments or run any of the steps of any of the previous or subsequent system embodiments.

E1. A system comprising:

one or more data processors; and

a non-transitory computer readable storage medium containing instructions which, when executed on the one or more data processors, cause the one or more data processors to perform processing to perform any of the previous or subsequent method embodiments or run any of the steps of any of the previous or subsequent method embodiments.

F1. A system comprising:

one or more data processors; and

a non-transitory computer readable storage medium containing instructions which, when executed on the one or more data processors, cause the one or more data processors to perform processing comprising: (a) obtaining a dried sample from a subject; (b) extracting the analyte of interest from the dried sample; and (c) detecting the analyte of interest extracted from the dried sample.

G1. A computer-program product tangibly embodied in a non-transitory machine-readable storage medium, including instructions configured to cause one or more data processors to perform processing comprising any of the previous method embodiments.
H1. A computer-program product tangibly embodied in a non-transitory machine-readable storage medium, including instructions configured to cause one or more data processors to perform processing comprising: (a) obtaining a dried sample from a subject; (b) extracting the analyte of interest from the dried sample; and (c) detecting the analyte of interest extracted from the dried sample.

That which is claimed is:

1. A method for measuring an analyte of interest in a dried sample comprising: (a) obtaining a dried sample from a subject; (b) extracting the analyte of interest from the dried sample; and (c) detecting the analyte of interest extracted from the dried sample.

2. The method of claim 1, wherein the analyte of interest is an analyte specific to SARS-CoV-2.

3. The method of claim 2, wherein the SARS-CoV-2 specific analyte is an antibody to SARS-CoV-2.

4. The method of claim 1, wherein the dried sample is a dried blood spot.

5. The method of claim 1, wherein the dried sample is dried plasma.

6. The method of claim 1, further comprising providing a cutoff index (COI) indicative of whether the subject has a detectable amount of the analyte and so is defined as positive, or does not contain a detected amount of the analyte and so is defined as negative.

7. The method of claim 6, wherein the COI is calculated to incorporate dilution of the sample that occurs during extraction of the SARS-CoV-2 specific analyte from the dried sample.

8. The method of claim 7, wherein the measured value is divided by a fractional cutoff to account for the dilution so as to provide a normalized COI value.

9. The method of claim 8, wherein the normalized COI comprises a cutoff value for serum.

10. The method of claim 6, wherein the COI for a positive sample is ≥1.0 and the COI for a negative sample is <1.0.

11. The method of claim 6, further comprising providing a COI range indicative that the sample is not determinate as being either positive or negative for the analyte of interest.

12. The method of claim 11, wherein the COI for a positive sample is greater than 2.0, the COI for a negative sample is less than 0.65 and the COI for an indeterminate sample is ≥0.65 COI and ≤2.0 COI.

13. The method of claim 1, wherein the analyte of interest is an antibody, and the antibody is measured using a sandwich assay employing a first antigen labeled with a detectable moiety and a second antigen labeled with a binding agent.

14. The method of claim 13, wherein the detectable moiety on the first antigen is an electrochemical moiety,

15. The method of claim 13, wherein the binding agent is streptavidin.

16. The method of claim 15, wherein the first antigen:antibody:second antigen complex is bound to a biotin-labeled electrode such that application of a voltage results in a chemiluminescent emission.

17. A system comprising:
one or more stations or components for: (a) obtaining a dried sample from a subject; (b) extracting the analyte of interest from the dried sample; and (c) detecting the analyte of interest extracted from the dried sample.

18. The system of claim 17, further comprising one or more data processors; and
a non-transitory computer readable storage medium containing instructions which, when executed on the one or more data processors, cause the one or more data processors to perform processing comprising: (a) obtaining a dried sample from a subject; (b) extracting the analyte of interest from the dried sample; and (c) detecting the analyte of interest extracted from the dried sample.

19. A computer-program product tangibly embodied in a non-transitory machine-readable storage medium including instructions configured to cause one or more data processors to perform processing comprising: (a) obtaining a dried sample from a subject; (b) extracting the analyte of interest from the dried sample; and (c) detecting the analyte of interest extracted from the dried sample.
